(19) United States
(12) Patent Application Publication
(10) Pub. No.: US 2019/0168019 A1
(43) Pub. Date: Jun. 6, 2019

(54) CONTROLLABLE LASER ACUPUNCTURE DEVICE

(71) Applicant: I-SHOU UNIVERSITY, Kaohsiung (TW)

(72) Inventors: Chih-Yu Wang, Kaohsiung (TW); Shu-Chen Chang, Kaohsiung (TW); Che-Chang Kuo, Kaohsiung (TW)

(21) Appl. No.: 15/878,482

(22) Filed: Jan. 24, 2018

(30) Foreign Application Priority Data
Dec. 1, 2017 (TW) 106142251

(51) Int. Cl. A61N 5/06; A61H 39/00

(52) U.S. Cl. CPC A61N 5/0619; A61N 2005/067; A61H 2039/005; A61H 39/00

(57) ABSTRACT

A controllable laser acupuncture device includes a laser generation module and a control unit. The laser generation module includes an emission end, a primary emission head mounted on the emission end, and a plurality of auxiliary emission heads mounted on the emission end. The control unit is electrically connected to the laser generation module. The control unit is configured to control each of the primary emission head and the plurality of auxiliary emission heads to emit or not to emit a laser beam and is configured to adjust properties of the laser beams outputted by the primary emission head and the plurality of auxiliary emission heads.

### CROSS REFERENCE TO RELATED APPLICATION

The application claims the benefit of Taiwan application serial No. 106142251, filed on Dec. 1, 2017, and the entire contents of which are incorporated herein by reference.

### BACKGROUND OF THE INVENTION

### 1. Field of the Invention

The present invention relates to a controllable laser acupuncture device and, more particularly, to a controllable laser acupuncture device using laser beams to simulate acupuncturing effects.

### 2. Description of the Related Art

Acupuncture therapy originates from China and is an alternative therapy having a long history. Thin metal needles are inserted into acupoints of a patient to make the patient feel sore, pain, or numbness, thereby providing a therapeutic effect or mitigating diseases. During the acupuncture therapy, a person can apply plural acupuncture techniques according to the body conditions and demands of the patient. Frequently used acupuncture techniques include lifting-thrusting and twirling. Lifting-thrusting includes inserting a thin metal needle into the human body and then slightly moving the needle along its longitudinal axis to shorten the time period for obtaining qi. Twirling includes rotating the thin metal needle about its longitudinal axis for gathering qi in the body meridians. Thus, twirling is an important skill in acupuncture therapy. The acupuncture therapy effect will be significantly decreased without twirling.

However, the traditional acupuncture therapy using thin metal needles is an invasive treating method that cannot avoid risks of infection and blood-borne diseases. Furthermore, the patient may experience physical pain and psychological fear while the thin metal pin is piercing his or her skin. Furthermore, the acupuncture therapy effect greatly relies on the experience of the person applying the needle, yet the same therapy effect cannot be guaranteed every time. In view of these concerns, laser acupuncture devices have been developed in recent years and use a laser beam to illuminate the skin of the patient to stimulate the acupoint, avoiding the risks of infection and achieving a painless therapy effect, which is very helpful in propagation of acupuncture therapy.

However, the conventional laser acupuncture devices can control the output intensity of only one laser beam, such that only the lifting-thrusting technique can be simulated. Namely, the twirling technique cannot be simulated. Improvement is, thus, required.

### SUMMARY OF THE INVENTION

To solve the above problems, an objective of the present invention is to provide a controllable laser acupuncture device capable of simulating both the lifting-thrusting technique and the twirling technique.

A controllable laser acupuncture device according to the present invention includes a laser generation module and a control unit. The laser generation module includes an emission end, a primary emission head mounted on the emission end, and a plurality of auxiliary emission heads mounted on the emission end. The control unit is electrically connected to the laser generation module. The control unit is configured to control each of the primary emission head and the plurality of auxiliary emission heads to emit or not to emit a laser beam and is configured to adjust properties of the laser beams outputted by the primary emission head and the plurality of auxiliary emission heads.

Thus, the controllable laser acupuncture device according to the present invention can simulate the lifting-thrusting technique and the twirling technique by adjusting the laser beams outputted by the primary emission head and the plurality of auxiliary emission heads, which can be stably practiced and reproduced without relying on the skill and experience of the user, providing an excellent, stable acupuncture therapy effect.

In an example, the plurality of auxiliary emission heads is mounted around the primary emission head. This uniformly increases the area of the skin of the human body illuminated by the laser beams while attaining a simulating effect of the twirling technique.

In an example, the plurality of auxiliary emission heads is mounted around the primary emission head and is spaced from each other by regular angular intervals. This permits easy adjustment of the plurality of auxiliary emission heads to provide various acupuncture effects.

In an example, the primary emission head is located in a central portion of the emission end. This increases the acupoint illumination accuracy and operational convenience under control of the laser beams.

In an example, an area of the laser beam outputted by the primary emission head is larger than an area of the laser beam outputted by each of the plurality of auxiliary emission heads. This increases the simulated acupuncture therapy effect.

In an example, the controllable laser acupuncture device further includes a pen-shaped housing. The laser generation module and the control unit are mounted in the pen-shaped housing. The emission end of the laser generation module is exposed outside of the pen-shaped housing. This permits easy operation and easy carriage of the whole controllable laser acupuncture device.

In an example, the controllable laser acupuncture device further includes a human machine interface mounted to an outer face of the pen-shaped housing. The human machine interface is electrically connected to the control unit. This increases the utility.

The present invention will become clearer in light of the following detailed description of illustrative embodiments of this invention described in connection with the drawings.

### BRIEF DESCRIPTION OF THE DRAWINGS

FIG. 1 is a partial, perspective view of a controllable laser acupuncture apparatus of an embodiment according to the present invention.

FIG. 2 is an end elevational view of the controllable laser acupuncture apparatus of FIG. 1.

FIG. 3 is a diagrammatic, partial, side view illustrating use of the controllable laser acupuncture device of FIG. 1.

### DETAILED DESCRIPTION OF THE INVENTION

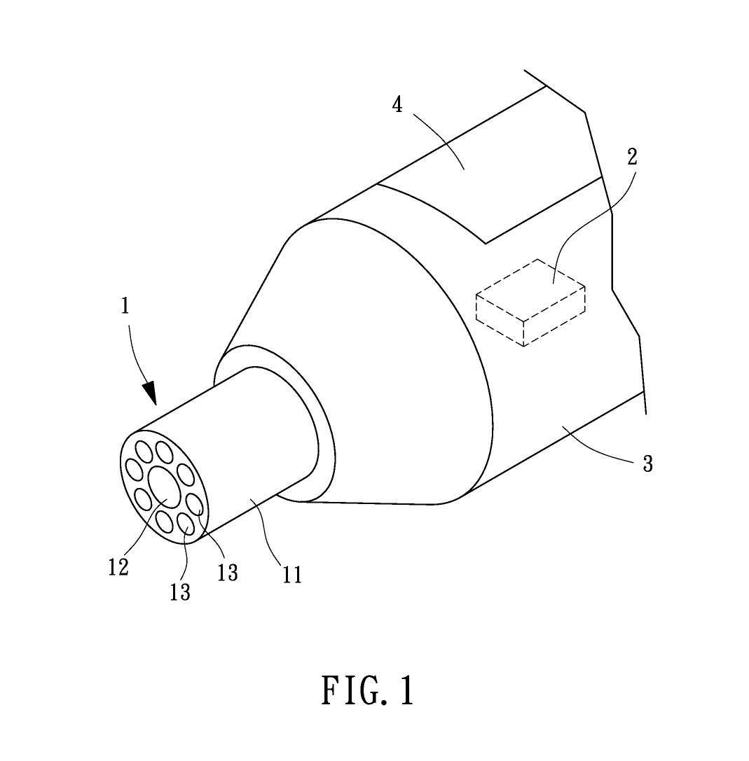

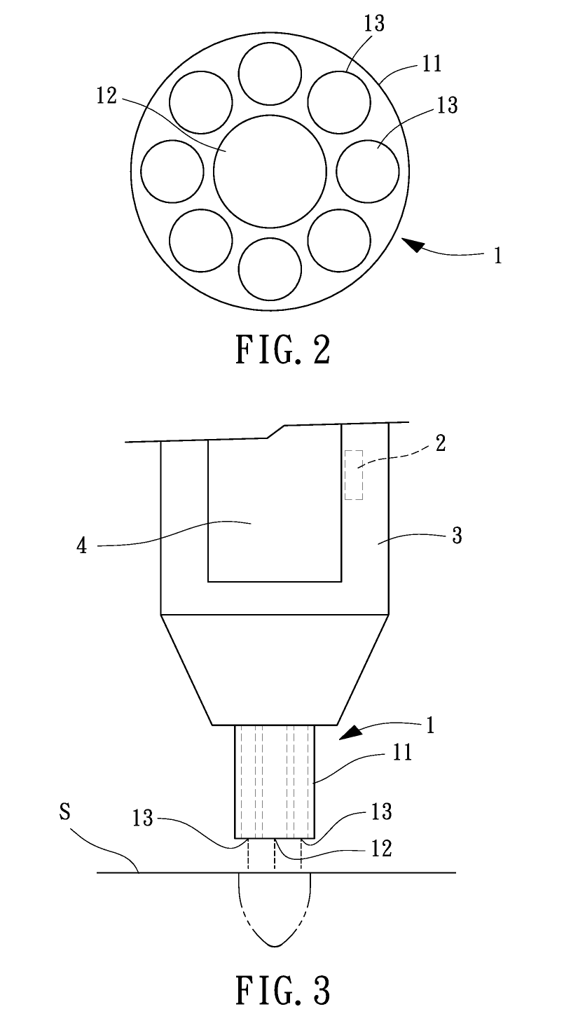

FIGS. 1 and 2 show a controllable laser acupuncture device of an embodiment according to the present invention. The controllable laser acupuncture device includes a laser generating module 1 and a control unit 2 electrically connected to the laser generating module 1.

The laser generating module 1 can be of any desired type and is configured to generate and project laser beams. The laser generating module 1 includes an emission end 11, and the laser beams generated by the laser generating module 1 are projected towards the emission end 11. The laser generating module 1 further includes a primary emission head 12 mounted on the emission end 11 and a plurality of auxiliary emission heads 13 mounted on the emission end 11. The primary emission head 12 and the plurality of auxiliary emission heads 13 can emit laser beams at the same time or in turns. When the laser beams illuminate the skin of a human body, the laser energy can penetrate the skin surface to simulate an acupuncture effect of a thin metal needle penetrating through the skin.

The control unit 2 is electrically connected to the laser generation module 1. The control unit 2 is configured to control each of the primary emission head 12 and the plurality of auxiliary emission heads 13 to emit or not to emit a laser beam and is configured to adjust properties (such as power, wavelength, and time) of the laser beams outputted by the primary emission head 12 and the plurality of auxiliary emission heads 13.

It is noted that in this embodiment, the plurality of auxiliary emission heads 13 is mounted around the primary emission head 12 to uniformly increase the area of the skin of the human body illuminated by the laser beams. Alternatively, the plurality of auxiliary emission heads 13 is controlled to emit laser beams in turn. Thus, a simulating effect of twirling a thin metal needle in an acupoint of the human body can be attained. The plurality of auxiliary emission heads 13 can be mounted around the primary emission head 12 and is spaced from each other by regular angular intervals. This permits easy adjustment of the plurality of auxiliary emission heads 13 to provide various acupuncture effects. The primary emission head 12 can be located in a central portion of the emission end 11 to increase the acupoint illumination accuracy and operational convenience under control of the laser beams. Furthermore, the area of the laser beam outputted by the primary emission head 12 can be larger than the area of the laser beam outputted by each of the plurality of auxiliary emission heads 13. Thus, the laser beam emitted from the primary emission head 12 simulates a thin metal needle per se, and the laser beams emitted from the plurality of auxiliary emission heads 13 simulate the stimulating effect provided by axial movement or rotation of a thin metal needle on an acupoint, increasing the simulated acupuncture therapy effect.

Furthermore, the controllable laser acupuncture device of this embodiment further includes a pen-shaped housing 3 and a human machine interface 4. The laser generating module 1 and the control unit 2 are mounted in the pen-shaped housing 3. The emission end 11 of the laser generating module 1 is exposed outside of the pen-shaped housing 3. The human machine interface 4 is mounted to an outer face of the pen-shaped housing 3 and is electrically connected to the control unit 2. Thus, the pen-shaped housing 3 permits easy operation and easy carriage of the whole controllable laser acupuncture device. The human machine interface 4 permits the user to activate different acupuncture modes or to set various control parameters, increasing the utility.

With reference to FIG. 3, in use of the controllable laser acupuncture device of this embodiment on a patient for acupuncture therapy, the user holds the pen-shaped housing 3 with the emission end 11 of the laser generating module 1 facing the skin S of a patient. Nevertheless, the emission end 11 is preferably spaced from the skin S of the patient to avoid infection. Then, a desired acupuncture mode can be selected through the human machine interface 4, and the control unit 2 can be operated to control whether to emit laser beams through the primary emission head 12 and the plurality of auxiliary emission heads 13 and to adjust the properties of the outputted laser beams, thereby achieving the desired acupuncture therapy effect.

For example, the control unit 2 can control the primary emission head 12 to emit a laser beam and can control the plurality of auxiliary emission heads 13 not to emit laser beams, which simulates penetration of a thin metal needle into the skin S of the patient. Furthermore, the intensity of the laser beam outputted by the primary emission head 12 can be controlled to increase or decrease gradually to thereby simulate the lifting-thrusting technique.

Furthermore, the primary emission head 12 can be controlled to continuously emit the laser beam, and the plurality of auxiliary emission heads 13 is controlled to emit laser beams in turn and in a fixed direction. Thus, the plurality of auxiliary emission heads 13 simulates twirling of a thin metal needle in the fixed direction, providing a simulated acupuncture effect of the twirling technique. Namely, a simulation of a left-turn twirling (the thumb moves forward and the index finger moves rearward) is achieved when the plurality of auxiliary emission heads 13 is controlled to emit laser beams in turn and in a rotating direction, attaining a “reinforcement effect” of acupuncture. On the other hand, a simulation of a right-turn twirling (the index finger moves forward and the thumb moves rearward) is achieved when the plurality of auxiliary emission heads 13 is controlled to emit laser beams in turn and in another rotating direction, attaining a “reduction effect” of acupuncture.

Furthermore, since the “reinforcement effect” of acupuncture requires a small twirling angle, a small force, a slow frequency, and a short operation time while the “reduction effect” of acupuncture requires a large small twirling angle, a large force, a fast frequency, and a long operation time, the controllable laser acupuncture device according to the present invention can use the control unit 2 to adjust the timing and properties of the laser beams outputted by the plurality of auxiliary emission heads 13, easily achieving the desired acupuncture effect while avoiding adverse influence on the laser acupuncture therapy effect affected by the experience of the user.

Furthermore, in addition to controlling the primary emission head 12 to continuously emit a laser beam, the controllable laser acupuncture device according to the present invention can control some of the plurality of auxiliary emission heads 13 on the side same to continuously emit laser beams and can control the remaining auxiliary emission heads 13 on the other side not to emit laser beams or to reduce the output energy of the laser beams on the other side, all of which can be attained without moving the pen-shaped housing 3. Thus, the overall area of the skin S of the patient illuminated by the laser beams can deviate to produce a local reinforcing and stimulating effect. Alternatively, two or more adjacent auxiliary emission heads 13 can be controlled to emit laser beams every time to thereby increase the stimulated area while proceeding with the twirling technique.

In addition to the above example of control, the controllable laser acupuncture device according to the present invention can use the primary emission head 12 and the plurality of auxiliary emission heads 13 to produce a variety of changes in control, which simulates various acupuncture techniques and is, thus, able to completely replace thin metal needles for achieving the acupuncture therapy effect (or even surpass the acupuncture therapy effect attained by thin metal needles).

In view of the foregoing, the controllable laser acupuncture device according to the present invention can simulate the lifting-thrusting technique and the twirling technique by adjusting the laser beams outputted by the primary emission head 12 and the plurality of auxiliary emission heads 13, which can be stably practiced and reproduced without relying on the skill and experience of the user, providing an excellent, stable acupuncture therapy effect.

Thus since the invention disclosed herein may be embodied in other specific forms without departing from the spirit or general characteristics thereof, some of which forms have been indicated, the embodiments described herein are to be considered in all respects illustrative and not restrictive. The scope of the invention is to be indicated by the appended claims, rather than by the foregoing description, and all changes which come within the meaning and range of equivalency of the claims are intended to be embraced therein.

What is claimed is:

1. A controllable laser acupuncture device comprising:
a laser generation module including an emission end, a primary emission head mounted on the emission end, and a plurality of auxiliary emission heads mounted on the emission end; and
a control unit electrically connected to the laser generation module, wherein the control unit is configured to control each of the primary emission head and the plurality of auxiliary emission heads to emit or not to emit a laser beam and is configured to adjust properties of the laser beams outputted by the primary emission head and the plurality of auxiliary emission heads.

2. The controllable laser acupuncture device as claimed in claim 1, wherein the plurality of auxiliary emission heads is mounted around the primary emission head.

3. The controllable laser acupuncture device as claimed in claim 2, wherein the plurality of auxiliary emission heads mounted around the primary emission head and is spaced from each other by regular angular intervals.

4. The controllable laser acupuncture device as claimed in claim 2, wherein the primary emission head is located in a central portion of the emission end.

5. The controllable laser acupuncture device as claimed in claim 1, wherein an area of the laser beam outputted by the primary emission head is larger than an area of the laser beam outputted by each of the plurality of auxiliary emission heads.

6. The controllable laser acupuncture device as claimed in claim 1, further comprising a pen-shaped housing, wherein the laser generation module and the control unit are mounted in the pen-shaped housing, and wherein the emission end of the laser generation module is exposed outside of the pen-shaped housing.

7. The controllable laser acupuncture device as claimed in claim 6, further comprising a human machine interface mounted to an outer face of the pen-shaped housing, wherein the human machine interface is electrically connected to the control unit.
